(19) United States
(12) Patent Application Publication
(10) Pub. No.: US 2007/0059351 A1
(43) Pub. Date: Mar. 15, 2007

(54) Transdermal patches containing a nitric oxide-donor and a second active agent and associated methods

(76) Inventors: George Anthony Calvert Murrell, Sidney (AU); Robert Ang, New York, NY (US); Sven Jacobson, New York, NY (US); David Geliebter, Franklin Lakes, NJ (US)

Correspondence Address: THORPE NORTH & WESTERN, LLP., 8180 SOUTH 700 EAST, SUITE 200, SANDY, UT, 84070, US

(21) Appl. No.: 11/366,207

(22) Filed: Mar. 1, 2006

Related U.S. Application Data
(63) Continuation-in-part of application No. 10/967,707, filed on Oct. 15, 2004.
(60) Provisional application No. 60/512,070, filed on Oct. 17, 2003.

(51) Int. Cl. A61K 9/70; A61K 31/21; A61K 31/655

(52) U.S. Cl. 424/449; 424/608; 514/509; 514/149

(57) ABSTRACT

The present invention is drawn to a transdermal patch for the delivery of a nitric oxide-donor and a second active agent. The patch can comprise a backing layer and an active agent-containing composition which is supported at least in part by the backing layer. The active agent-containing composition can include an amount of a nitric oxide-donor and an amount of a second active agent. The transdermal patch can have a drug delivery zone defined by the area where the composition contacts an intact human skin site, and the transdermal patch can be formulated to deliver a nitric oxide donor, such as nitroglycerin, at from about 5 μg/hour to about 85 μg/hour. The second active agent can be selected from a number of agents including NSAIDS, opioids, local anesthetics, menthol, salicylic acid, salicylic acid derivatives, vanilloid receptor-1 activators, corticosteroids, vasoconstrictors, and combinations thereof.

The present application is a continuation-in-part of U.S. patent application Ser. No. 10/967,707, filed Oct. 15, 2004, which claims the benefit of U.S. Provisional Patent Application Ser. No. 60/512,070, filed Oct. 17, 2003, each of which is incorporated herein by reference in their entireties.

### FIELD OF THE INVENTION

The present invention relates to methods and devices for transdermally co-delivering low doses of a nitric oxide-donor with other active agents. The devices can be used for variety of treatment regimens including pain relief, accelerated healing, and/or improved function of tendons afflicted with tendinopathy, including tendinosis and tendinitis.

### BACKGROUND OF THE INVENTION

Nitric Oxide (NO) is a highly reactive chemical species and is extremely short-lived, so NO is most typically supplied to a patient in the form of glyceryl trinitrate (GTN, also known as nitroglycerin) or through some other substance capable of generating NO (termed “nitric oxide donor”). Such donor substances tend to be more stable than NO itself and can thereby be used to release NO over time. NO donors can be administered sublingually (e.g., by tablets placed under the tongue), transdermally (e.g., by a dermal composition placed on the skin), or in other ways.

NO is produced endogenously by three isoforms of the enzyme nitric oxide synthase, inducible nitric oxide synthase (iNOS), an isoform originally found in endothelial cells (eNOS), and an isoform originally found in brain tissue and neuronal cells (bNOS). NO is produced in large amounts by inflammatory cells such as macrophages, neutrophils, lymphocytes, and peripheral-blood monocytes during immunological reactions and septic shock. There is also an inducible form of nitric oxide synthase in cartilage.

NO is believed to act as a vasodilator and has been found to be useful in treating several disorders, most notably angina pectoris. NO has also been shown to provide enhanced or accelerated wound healing and relief of pain. Wound healing involves the recruitment of inflammatory cells, followed by fibroblasts, to the site of the wound, where collagen and other connective tissue elements are deposited. The collagen fibers then gradually realign to resemble the original connective tissue (e.g., tendon, ligament, skin, etc.). Topical NO donation has been used effectively to treat cutaneous wounds and tendons in animal models via mechanisms that may include stimulation of collagen synthesis in fibroblasts.

Though NO can be used in treating a variety of ailments and conditions, there is room for improvement in the area of providing new NO formulations for more effective treatment regimens for various ailments.

### SUMMARY OF THE INVENTION

The present invention is drawn to devices and methods for transdermally delivering a nitric oxide-donor in combination with other active agents to a subject. In one embodiment a transdermal patch for the delivery of a nitric oxide donor and a second active agent, can include a backing layer and an active agent-containing composition. The active agent-containing composition can be supported at least in part by the backing layer and can include a nitric oxide-donor and a second active agent selected from the group consisting of menthol, vanilloid receptor-1 activators, salicylic acid, derivatives thereof, and mixtures thereof. The active agent-containing composition can be formulated to contain from about 1 wt % to about 20 wt % of a nitric oxide donor.

In another embodiment, a transdermal patch for the delivery of a nitric oxide donor and a second active agent can include a backing layer and an active agent-containing composition. The active agent-containing composition can be supported at least in part by the backing layer and can include a nitric oxide-donor and a second active agent selected from the group consisting of opioids, local anesthetics, NSAIDS, and mixtures thereof. The active agent-containing composition can be formulated to contain from about 1 wt % to about 20 wt % of a nitric oxide donor.

In another embodiment, a transdermal patch for the delivery of a nitric oxide donor and a second therapeutic agent can include a backing layer and an active agent-containing composition. The active agent-containing composition can be supported at least in part by the backing layer and can include a nitric oxide-donor and a corticosteroid. The active agent-containing composition can be formulated to contain from about 1 wt % to about 20 wt % of a nitric oxide donor.

In yet another embodiment, a transdermal patch for the delivery of a nitric oxide-donor and a second therapeutic agent can include a backing layer and an active agent-containing composition. The active agent-containing composition can be supported at least in part by the backing layer and can include a nitric oxide-donor and a vasoconstrictor. The active agent-containing composition can be formulated to contain from about 1 wt % to about 20 wt % of a nitric oxide donor.

In still another embodiment, a method for delivering a nitric oxide donor and a second active agent to a subject in need thereof can include applying a transdermal patch to a skin surface of said subject. The transdermal patch can include an active agent-containing composition including a nitric oxide donor and a second active agent selected from the group consisting of menthol, a vanilloid receptor-1 activator, salicylic acid, an opioid, a local anesthetic, an NSAID, a corticosteroid, a vasoconstrictor, derivatives thereof, and mixtures thereof. The active agent-containing composition can be formulated to contain from about 1 wt % to about 20 wt % of a nitric oxide donor.

Exemplary subjects in need thereof i) may be experiencing pain and the transdermal patch is applied for reducing this pain, ii) may have damaged tissue and the transdermal patch is applied for accelerating healing of the damaged tissue, iii) may have tendinopathy (tendinosis or tendonitis) and the transdermal patch is applied to improve function of an afflicted tendon iv) may have cancer, v) may be suffering from acute inflammation, and/or vi) may have other physical ailments.

### DETAILED DESCRIPTION OF THE PREFERRED EMBODIMENT(S)

Before particular embodiments of the present invention are disclosed and described, it is to be understood that this invention is not limited to the particular process and materials disclosed herein and as such may vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and, “the” include plural referents unless the context clearly dictates otherwise. Thus, for example, reference to an active agent-containing composition including “an opioid” includes one or more opioids and reference to “the second active agent” includes reference to one or more second active agents.

As used herein, “subject” refers to a mammal that may benefit from the administration of the systems or methods of this invention. Examples of subjects include humans, and may also include other animals such as horses, pigs, cattle, dogs, cats, rabbits, and aquatic mammals.

As used herein, the terms “formulation” and “composition” are used interchangeably and refer to mixtures, solutions, dispersions, etc. of two or more compounds, elements, or molecules.

As used herein, the term “nitric oxide donor” or “NO-donor” refers a compound or mixture of compounds which, when delivered to a subject, increases the concentration of nitric oxide present in the subject. Nitroglycerin is a preferred nitric oxide donor.

As used herein, the term “drug delivery zone” refers to the area of skin which comes into direct contact with the portion of the transdermal patch which delivers the active agents, e.g. the NO-donor and the second active agent. For example, when the delivery device is a transdermal matrix patch the drug delivery zone would be the area in which the active agent-containing composition contacts the skin.

The term “active agent-containing composition” refers to a composition that contains both the NO-donor and the second active agent. The active agent-containing composition can take different forms depending on the nature and type of the transdermal patch. For example, when the transdermal patch is an adhesive matrix patch, the active agent containing composition will be the adhesive matrix. When the transdermal patch is a reservoir patch, the active agent-containing composition will be a liquid or semi-solid contained within the patch.

As some NO-donors may cause undesirable tolerance issues, in some cases it can be desirable allow for drug holidays. “Drug holiday(s)” refers to periods of time in which the transdermal patch is removed for a predetermined length of time before a subsequent patch is administered. For example, a patch of the present invention may be applied to the skin of a subject for a period of 12 hours after which the patch is removed and a drug holiday period of 12 hours is allowed to pass before a subsequent patch is applied to the subject's skin. Other periods of time for drug delivery and drug holidays can also be implemented, as would be known to those skilled in the art after considering the present disclosure.

As used herein the term “continuous” or “continuously” in the context of drug administration refers to regular or constant dosing of predetermined amounts of a NO donor to a subject. For the purposes of the present invention the incorporation of planned regular drug holidays into a dosing regimen does not destroy the continuous nature of the drug administration period. Continuous drug administration also covers periods where a new transdermal patch is applied directly after the removal of a used patch without a drug holiday.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity, and thus, should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

NO can be locally delivered to and beneficial to a site for a variety of reasons including providing pain relief, improving wound healing, improving function, reducing inflammation, treating or preventing angina, inhibiting cancer metastasis, etc. Nitric oxide donor compounds can also be delivered in combination with other active agents to augment or enhance the benefits associated with the increased NO. The local delivery of both the nitric oxide donor and the second active agent can be accomplished through the use of a transdermal patch which is placed on a skin surface proximate, distal, or over the site in need of treatment, e.g., painful or wounded site. As nitroglycerin, a preferred NO donor, is typically rapidly systemically absorbed and distributed, it is feasible that a patch of the present invention could be placed on a skin site which is remote from the site requiring treatment and still be effective for providing the desired outcome. However, it is preferred that the transdermal patch be applied proximate the area for which pain relief, wound healing, or improved function is desired so as to allow for maximal effectiveness of the patch, particularly by the second active, but this is not required. To provide one example, NO delivery in accordance with embodiments of the present invention can be carried out to treat tendons suffering from tendinopathy, including either tendinosis or tendonitis.

For example, if an Achilles tendon is in need of pain relief, wound healing, or other treatment, a transdermal patch containing nitroglycerin and an NSAID may be placed on the skin proximate the Achilles tendon. In this embodiment, the patch delivers the active agents (both the nitroglycerin i.e. the first active agent, and the NSAID, i.e. the second active agent) through the skin increasing the NO concentrations and providing for reduced pain, reduced inflammation, accelerated wound healing, and/or improved function. Other examples of uses for the transdermal patch of the present invention include but are not limited to the enhancing healing of damaged muscles or reducing the pain associated therewith, enhancing healing and relieving pain of a chronic skin ulcer, treatment of heart failure, treatment of angina, tendonitis, tendinosis, prevention of cancer metastasis, prevention of thrombophlebitis, treatment of acute inflammation including inflammation associated with acute local arthritis and thrombophlebitis, accelerate bone healing, accelerate post-operative recovery, prevention of unnecessary inflammation, etc.

The second active agents for co-delivery with the NO-donor of the present invention can be a non-steroidal anti-inflammatory drugs (NSAID), salicylic acid or its derivatives, menthol, vanilloid receptor-1 activators, local anesthetics, opioids, corticosteroids, vasoconstrictors, or combinations thereof.

In one embodiment, the second active agent is menthol, a vanilloid receptor-1 activator, salicylic acid, derivatives thereof, or combinations thereof. Examples of vanilloid receptor-1 activators which can be used in as the second active agent include but are not limited to capsaicin, dihydrocapsaicin, nordihydrocapsaicin, homodihydrocapsaicin, homocapsaicin, resiniferatoxin, civamide, and combinations thereof. Non-limiting examples of salicylic acid derivatives which can be used as the second active agent include salicylamide, sodium salicylate, diflunisal, niclosamide, acetyl salicylic aci, choline magnesium trialicylate, hydroxyethylsalicylate, diethylamine salicylate, triethylamine salicylate methyl salicylate and combinations thereof.

In another embodiment, the second active agent can be an NSAID, opioid, local anesthetic, or combination thereof. Examples of NSAIDS which can be used as the second active agent include but are not limited to celecoxib, diclofenac potassium, diclofeniac sodium, diclofenac sodium with misprostol, diflunisal, etodolac, fenoprofen calcium, flurbiprofen, ibuprofen, indomethacin, ketoprofen, mclofenamate sodium, mefenamic acid, meloxicam, nabumetone, naproxen, naproxen sodium, oxaprozin, piroxicam, rofecoxib, salsalate, sulindac, tolmetin sodium valdecoxib, and combinations thereof. Examples of local anesthetics which can be used as the second active agent include but are not limited to benzocaine, mepivacaine, ropivacaine, bupivacaine, lidocaine, prilocaine, procaine, chloroprocaine, EMLA, lignicaine, tetracaine, levobupivacaine, and combinations thereof. Examples of opioids which can be used as the second active agent include but are not limited to morphine, oxycodone, hydrocodone, codeine, diamorphine, dihydrocodeine, oxymorphone, nicomorphine, methadone, levomethadyl acetate hydrochloride, pethidine, fentanyl, alfentanil, sufentanil, remifentanil, ketobemidone, carfentanyl, propoxyphene, dextropropoxyphene, dextromoramide, bexitramide, piritramide benzomorphan derivatives, pentazocine, phenazocine, burprenorphine, butorphanol, nalbudine, dezocine, etorphine, tilidine, tramadol, loperamide, diphenoxylate, naloxone, naltrexone, and combinations thereof.

In yet another embodiment, the second active agent is a corticosteroid. Examples of corticosteroids which can be used as the second active agent include but are not limited to prednisone, prednisolone, beclomethasone, cortisone, dexamethasone, hydrocortinsone, methylprednisolone, triamcinolone, fludrocortisones, deflazacort, beclomethasone, dexamethasone, cortisol, and combinations thereof.

In still another embodiment, the second active agent is a vasoconstrictor. Non-limiting examples of vasoconstrictors which can be used as the second active agent include but are not limited to epinephrine, norepinephrine, levonordefrin, felypressin, phenylephrine, metaraminol, flunarizine, hydroxynorephedrine, methoxamine HCl, pizotifen, propranolol, ergotamine, caffeine, sumatriptan succinate, and combinations thereof.

There are significant advantages of applying the nitroglycerin and the second active agent using to a skin site using a transdermal patch rather than a cream or ointment. One advantage is that transdermal patches can provide measured sustained release of active agents over a desired period of time. Another benefit is that a patch can be easily removed in the event that a patient experiences unwanted side-effects associated with the patch. A further advantage of using a transdermal patch relates to the ability to deliver more precise dosages. Although creams and ointments can be prepared to contain specific concentrations active agents, they can be applied at any thickness and over any area of skin yielding inconsistent dosing. This inconsistent dosing can lead to over dosing or under dosing of the active agents. For example, overdosing of nitroglycerin can result in unwanted side effects including severe skin irritation, headaches, or vascular problems including hypotension. Overdosing of the second active agent can also cause dangerous and unwanted side effects. For example, overdosing of fentanyl or other opioids can pose serious health risks including death. Under dosing can result in ineffective treatment.

In accordance with one particular, non-limiting embodiment, it has been discovered that by more precise dosing to the tissue using a patch that delivers low doses of nitroglycerin over a larger surface area, which is co-delivered with therapeutically effective amounts of the second active agent, irritation can be reduced, skin patch adherence is improved, and appropriate amounts of nitroglycerin can be delivered over a larger surface area where needed. Larger areas of attachment, or larger drug delivery zones, also have the added benefit when the application site is to a mobile joint, e.g., elbow, knee ankle, etc, or an area in which large amounts of rubbing or contact can occur e.g. back, legs, arms, etc. As afflicted areas are often joints, the transdermal patch may be of a size that makes it easy to apply and remove when desired, and further, the patch can be manufactured to be a size that is more likely to stay affixed to a joint while still delivering low doses of an NO-donor. In other words, it has been recognized that more precise low doses can treat various tissue ailments and provide the significant advantages associated with delivering these low dosages over drug delivery zones that are larger in area per dosage delivered than those previously known.

The transdermal patches of the present invention can deliver a dose of nitroglycerin, or other NO donor, and a dose of a second active agent which together are pharmaceutically effective for promoting beneficial results, such as pain relief, wound healing, improving function, while reducing or eliminating the risks and inconveniences of traditional nitroglycerin patches. The patches of the present invention can be sized for convenient application and removal to all areas of the body, but in particular to areas where there are large amounts of skin stretch or rubbing, e.g. joints. For all sizes of patches of the present invention, the minimum delivery zone size is 2.5 cm². In a preferred embodiment the drug delivery zone can be at least 5 cm².

There are a wide variety of nitric oxide donors which can be used in the present invention. Examples of nitric oxide donors include but are not limited to nitroglycerin, isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof. In a preferred embodiment, the nitric oxide donor is nitroglycerin.

As mentioned, a preferred NO donor is nitroglycerin or glyceryl trinitrate (GTN, also called 1,2,3-propanetriol trinitrate). Nitroglycerin is exemplary of one preferred NO-donor for use in accordance with embodiments of the present invention. The transdermal patch of the present invention can provide for reduced pain, wound healing, or improved function by delivering the nitric oxide donor at dosage rates of from about 5 μg/hour to about 85 μg/hour. Further, in one embodiment, the NO-donor release rate can be from about 10 μg/hour to about 60 μg/hour. In one embodiment the nitric oxide donor is present in the active agent-containing composition in an amount of from 0.1 wt % to 60 wt %. In yet another embodiment the nitric oxide donor is present in an amount from 3 wt % to 35 wt %. In another embodiment, the nitric oxide donor is nitroglycerin and is used in the formulation of a transdermal matrix patch. In one embodiment, the nitroglycerin comprises from about 1 wt % to about 25 wt % of the matrix in the transdermal matrix patch. In yet a further embodiment, nitroglycerin comprises from about 6 wt % to about 18 wt % of the matrix in the transdermal matrix patch.

The amount of the second active agent present the active agent-containing composition can be dependent a number of factors including the desired type and potency of the second active agent, the type of patch, as well as the desired result. Generally, second active agent will be present in the active agent containing composition in at from about 0.01 wt % to about 40 wt %. In another embodiment the second active agent can be present in the active agent-containing composition in an amount of 0.5 wt % to 35 wt %. When the second active agent is menthol, it can be present in the active agent-containing composition at from about 1 wt % to about 20 wt %. When the second active agent is salicylic acid or a derivative thereof, it can be present in the active agent containing composition in an amount of from about 2 wt % to about 30 wt %. When the second active agent is a vanilloid receptor-1 activator, it can be present in the active agent composition in an amount of from about 0.01 wt % to about 10 wt %. When the second active agent is an NSAID, it can be present in the active agent containing composition in an amount of from about 3 wt % to about 25 wt %. When the second active agent is a local anesthetic, it can be present in the active agent containing composition in an amount of from about 0.5 wt % to about 10 wt %. When the second active agent is an opioid, it can be present in the active agent containing composition in an amount of from about 0.5 wt % to about 10 wt %. When the second active agent is a corticosteroid, it can be present in an amount of from about 0.5 wt % to about 10 wt %. When the second active agent is a vasoconstrictor, it can be present in an amount of from about 0.5 wt % and about 20 wt %.

A unique feature of the present invention is that the patches have a low delivery rate of NO-donor over the entire patch, and in one embodiment, as a rate per unit area (per cm²) of the drug delivery zone as compared with commercially available NO-donor patches. In one embodiment, the reduced delivery rate per unit area over the drug delivery zone allows for a patch with a relatively large drug delivery zone to deliver relatively small amounts of nitroglycerin or other NO-donor in combination with the second active agent.

This being stated, in one embodiment, the patches of the present invention can provide a delivery rate of the nitric oxide donor to the drug delivery zone in amounts of from about 1 μg/cm²/day to about 600 μg/cm²/day. In another embodiment, the patches can deliver from about 1 μg/cm²/day to about 280 μg/cm²/day. In yet another embodiment the patches can deliver from 10 μg/cm²/day to about 280 μg/cm²/day. In a further embodiment the patches can deliver from about 50 μg/cm²/day to about 250 μg/cm²/day. The lower delivery rates of the nitric oxide donor allow for the increase in the patch's drug delivery zone without increasing the dosage amount delivered to the patient. The lower dosage amounts can also decrease or eliminate some of the side effects which are affiliated with high dosages of nitroglycerin, namely headache, lightheadedness, and hypotension. This being stated, low dosages of nitroglycerin over larger surface areas is only exemplary of one embodiment of the present invention. Other dosages and other delivery zones can also be implemented in accordance with embodiments of the present invention.

As described above, each patch of the present invention has an area, known as the drug delivery zone, which is defined to be the area where the active agent-containing composition contacts an intact human skin surface. The area of the drug delivery zone can vary depending on the desired rate of delivery per cm² of the drug delivery zone and the total dosage amount to be delivered in a given dosing period. The size of the drug delivery zone can be from about 2.5 cm² to about 100 cm². In another embodiment, the size of the drug delivery zone is from about 3 cm² to about 50 cm².

The patches of the present invention can be used for administering NO-donors and the second active agent for both short and long periods of time. For reduced pain, wound healing, and improved function, the transdermal patch can be formulated to be able to sustain delivery of nitroglycerin (or other NO donor) over a continuous period of time of from 4 hours to 7 days. In another embodiment, the transdermal patch is formulated to deliver the nitric oxide donor for a continuous period of from about 1 to about 3 days. In a further embodiment the transdermal patch is formulated to deliver the nitric oxide donor for a continuous period of from about 12 hours to 24 hours. Patches can also be continuously administered over an administration period of from about 1 week to about 1 year. In one embodiment, a continuous administration period can last from about 1 day to about 24 weeks. As stated above, for the purposes of the present invention, planned regular drug holidays can be incorporated into an administration period without destroying its continuous nature.

The relationship between the total NO-donor content of a patch and the amount of NO-donor that is actually delivered to the skin depends in large part on the adhesive and other materials used in the patch. This relationship is discussed in, for example, U.S. Pat. Nos. 4,954,344; 4,849,226; 4,812,313; and 5,186,938, which to the extent compatible with the teachings of the present invention are incorporated herein by reference.

In addition to the active agents, the active agent-containing composition of the present invention can also include various binders and excipients as are well known in the transdermal patch arts. Examples include, but are not limited to solvents, permeation enhancers, and crosslinkers. Examples of permeation enhancers include but are not limited to polyethylene glycols, surfactants, and combinations thereof.

The transdermal patches of the present invention can take a wide variety of structural forms, including reservoir patches and matrix patches. In the broadest sense, all patches include an outer layer (or “backing layer”) that is distal to the skin (except where used to attach to the skin around the periphery of the active agent-containing portion of the patch). The backing layer protects the active agent-containing portion of the patch from the outside environment. A matrix patch includes a drug-in-adhesive layer that is typically attached to the backing layer and which contacts the skin. In matrix patches, the drug and adhesive can be mixed more or less homogenously, or alternatively, the drug and adhesive can be discretized with one or more “islands” of drug. A reservoir patch typically includes a reservoir of drug where the reservoir is defined by the backing layer and a permeable layer of material that contacts the skin and allows the drug to pass there through. Both of these types of transdermal patches are well known in the art. In either case, both types of patches have a backing layer which supports, in some way, an active agent-containing composition in accordance with embodiments of the present invention.

The backing layer is typically made of plastic or other resilient material and may be impermeable to gas and/or liquid. For patches that are placed on “active” skin regions (e.g., portions of skin that are near or that overlie joints, so that the skin is subject to occasional or frequent stretching or deformation), the backing layer can be formed of a material that is dimensioned and balanced appropriately to meet the need for flexibility (so that the patch does not substantially impede the joint flexing or extending motion) with the need for toughness to resist breakage or other failure.

With specific reference to the types of adhesives that can be present in matrix transdermal patches, or which can be applied to a porous membrane often used for reservoir patches, a wide variety of pharmaceutically-acceptable adhesive polymers can be used in connection with the present invention. Non-limiting examples of adhesives which can be used in the patches of the present invention include acrylic adhesives, polyacrylic adhesive polymers, acrylate copolymers (e.g., polyacrylate), silicone-based adhesives, polyisobutylene adhesive polymers, and combinations thereof. The adhesive matrix can contain varying amounts of the nitric oxide donor depending on the particular donor and the desired dosage and delivery rates.

### EXAMPLES

The following examples illustrate exemplary embodiments of the invention. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what is presently deemed to be practical embodiments of the invention.

### Example 1

Prototype transdermal patches containing a nitric oxide-donor in the form of nitroglycerin and a second active agent are formulated to contain about 6 wt % to about 18 wt % nitric oxide-donor, 0.01 wt % to about 40 wt % of a second active agent, and about 40 wt % to about 94 wt % of an acrylic adhesive (DuroTak 87-2194). The general preparation of the patches involves the following steps:

1. The nitric oxide-donor and second active agent are diluted in the DuroTak 87-2194 adhesive and ethyl acetate solvent forming the drug solution.

2. The drug solution of the adhesive blend is formed onto a release liner using a mechanical coater.

3. The coated release liner is then passed through an oven which causes the solvent (e.g. ethyl acetate and any solvent present in the liquid DuroTak) to evaporate, forming a solid, tacky layer of adhesive matrix that contains nitroglycerin dispersed in an adhesive matrix.

4. A polyethylene film is then laminated to the adhesive matrix.

5. The active agent-containing patch laminate is then cut to specified dimensions using a die cutter and the patches are then individually pouched in sealed foil-lined material.

The transdermal patches of the present invention can be formulated according to the above percentages to provide a various delivery rates, both for the nitric oxide-donor and the second active agent. Based on the above listed percentages and the present disclosure, one skilled in the art would readily be able to formulate the patches of the present invention after considering the present disclosure. It is worth noting that the delivery rates of the second active agents generally have greater variation due to the different types (e.g., local anesthetic, opioid, NSAID, salicylic acid derivates, etc.) and potencies of each active agent. As the second active agents of the present invention are known in the art, the physiologically safe values are known and are used in determining appropriate percentages, and thereby appropriate delivery rates, for each second active agent.

The patches taught in Examples 2 to 16 are formulated and manufactured as described in this example. To achieve the delivery rates set forth therein, the ratios and/or concentrations of the nitroglycerin and the second active agent can be determined using routine experimentation.

### Example 2

A transdermal patch containing 6 wt % nitroglycerin and 10 wt % menthol is formulated as disclosed in Example 1. The patch is applied to a skin site proximate an afflicted Achilles tendon of a human subject experiencing pain affiliated with tendinopathy. The patch has a drug delivery zone of about 7.2 cm². After 24 hours, the patch is removed and replaced with an identical patch. The patch is replaced once daily for a period of two weeks at which time the pain associated with the tendinopathy is reduced.

### Example 3

Same as Example 2, except that the administration period is for 8 weeks.

### Example 4

Same as Example 2, except that the patch is removed after the 12 hour administration period and the subject does not reapply a second patch until after the occurrence of a 12 hour drug holiday period. After the drug holiday, a new patch is applied. This administration period is continued for a period of 6 weeks.

### Example 5

Same as example 1, except the patch contains 20 wt % menthol.

### Example 6

A transdermal matrix patch containing 8 wt % nitroglycerin and 0.1% capsaicin is formulated according to Example 1. The patch is applied to a skin site proximate an afflicted shoulder tendon of a subject. The patch has a drug delivery zone of about 100 cm². After an administration period of about 24 hours the patch is removed and a new patch applied in its place. This is repeated daily for a period of 4 weeks at which time the subject has reduced pain and tenderness associated with the afflicted tendon and improved function thereof.

### Example 7

A transdermal matrix patch containing 16 wt % nitroglycerin and 18 wt % methyl salicylate is formulated according to Example 1. The patch is applied to a skin site proximate to an afflicted elbow of a human subject experiencing pain affiliated with overuse extensor tendinopathy. After 24 hours, the patch is removed and a new patch is applied to a new skin site proximate the afflicted elbow. This pattern is repeated daily for a period of 24 weeks at which time the pain and tenderness associated with the tendinopathy is reduced.

### Example 8

Same as Example 7, except that the initial patch is removed after a period of 12 hours at which time a drug holiday period of 12 hours is allowed to pass and then a second patch is applied to or proximate the same skin site and left for a period of 12 hours. This is repeated for a period of 12 weeks or until the elbow is pain free.

### Example 9

Same as Example 7 except the patch contains 10 wt % nitroglycerin and 30 wt % methyl salicylate.

### Example 10

A transdermal matrix patch is formulated to contain 12 wt % nitroglycerin and about 5 wt % morphine HCI. The patch has a drug delivery zone of 7.2 cm². The patch is applied to a skin site proximate a skin ulcer of a human subject. After 24 hours, the patch is removed and replaced with a second similar patch. This cycle is repeated every 24 hours for 2 weeks or until the skin ulcer is at least substantially healed.

### Example 11

A transdermal patch is formulated to contain about 8% nitroglycerin and about 10 wt % of meloxicam. The patch is applied to a skin site proximate a surgically repaired Achilles tendon of a subject. The patch has a drug delivery zone of 20 cm². The patch is left on the skin site for a period of 24 hours, at which time it is replaced with a similar patch for another period of 24 hours. After 5 weeks of consecutive wearing of the patches, the subject experiences less pain associated with the surgically repaired tendon and has improved function thereof.

### Example 12

Same as Example 11, except the patch is formulate to contain about 3 wt % of meloxicam.

### Example 13

A transdermal patch is formulated to contain about 9.1% nitroglycerin and about 5 wt % of flurbiprofen as described in Example 1. The patch is applied to a skin site proximate an area of acute inflammation on a subject. The patch has a drug delivery zone of 2.5 cm². The patch is left on the skin site for a period of 48 hours, at which time it is replaced with a new patch. This process is repeated for a period of 2 weeks after which the acute inflammation is reduced.

### Example 14

A transdermal patch is formulated to contain 16 wt % nitroglycerin and 2.5 wt % of hydrocortisone according to Example 1. The patch is applied to a skin site of a patient suffering from tendinosis. The patch has a drug delivery zone of approximately 20 cm². The patch is applied to a skin site proximate an injured elbow tendon a human subject. After 24 hours, the patch is removed and replaced with a second similar patch. This cycle is repeated every 24 hours for 7 weeks or until the subject has reduced pain associated with the injured tendon and experiences improved function thereof.

### Example 15

Same as Example 13 except that the cycle is repeated every 12 hours for a period of 2 weeks.

### Example 16

A transdermal patch is formulated and made in accordance to Example 1 to contain 10 wt % nitroglycerin and 5 wt % prilocaine. The patch is applied to a skin site proximate a surgically repaired wound having associated inflammation and pain. The patch has a drug delivery zone of 2.5 cm². The patch is left on the skin site for a period of 4 hours, at which time it is removed and replaced with a new patch. This process is repeated for a period of 8 weeks after which the pain and inflammation associated with the surgically repaired wound are reduced.

### Example 17

A 35 year old male patient suffering from chronic tendinopathy of the left Achilles tendon applies a transdermal patch containing 4 wt % nitroglycerin and 0.5 wt % of capsaicin. The patch is left in place for a period of two weeks. The patient experiences a moderate decrease in tenderness and ankle soreness by day 2 of therapy, which progressively improves over the treatment period. Following the treatment period the patient feels his ankle is pain free. The ankle remains pain free for several weeks beyond the treatment period.

### Example 18

A 35 year old female patient suffering de Quervain's tendinopathy in the right extensor tendons of the thumb applies a transdermal patch containing 8 wt % nitroglycerin and 10 wt % of menthol. The patch is applied for a period of four weeks. This patient suffers this condition due to the arrival of a new baby and the consequent carrying as an unusual daily activity, and physical therapy and intermittent use of a wrist splint provides little relief of symptoms. The patient notices a decrease in pain within one day of beginning treatment, and a subsequent assessment by a physician at week four of treatment reveals no positive signs or symptoms of de Quervain's disease. This includes a negative Finklestein test. The patient remains pain free for several months post-treatment.

### Example 19

A 65 year old male patient suffering from chronic tennis elbow applies a transdermal patch containing 10 wt % nitroglycerin and 15 wt % methyl salicylate. The patch is left applied for a period of one week. The patient experiences a moderate decrease in pain upon elicitation at the end of the treatment period as assessed by grip strength and resisted wrist dorsiflexion. The patient remains with some residual symptoms, though the patient's symptoms are less severe than before treatment.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A transdermal patch for the delivery of a nitric oxide donor and a second active agent, comprising:
a backing layer, and
an active agent-containing composition supported at least in part by the backing layer, said active agent-containing composition comprising a nitric oxide donor and a second active agent selected from the group consisting of menthol, a vanilloid receptor-1 activator, salicylic acid, derivatives thereof, and mixtures thereof, said active agent-containing composition containing between 1 wt % and 20 wt % of nitric oxide donor.

2. The transdermal patch of claim 1, wherein the nitric oxide donor is selected from the group consisting of nitroglycerin, isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof

3. The transdermal patch of claim 1, wherein the nitric oxide donor is nitroglycerin.

4. The transdermal patch of claim 1, said transdermal patch having a drug delivery zone defined by the area where the active agent-containing composition contacts an intact human skin site, wherein the drug delivery zone has an area from about 2.5 cm² to 100 cm².

5. The transdermal patch of claim 1, said transdermal patch having a drug delivery zone defined by the area where the active agent-containing composition contacts an intact human skin site, wherein the drug delivery zone has an area from about 3 cm to about 50 cm².

6. The transdermal patch of claim 1, wherein the transdermal patch is an adhesive matrix patch.

7. The transdermal patch of claim 4, wherein the adhesive matrix includes an acrylic polymer.

8. The transdermal patch of claim 1, wherein the transdermal patch is formulated to deliver the active agents from the active agent-containing composition for a period of from 4 hours to 7 days.

9. The transdermal patch of claim 8, wherein the period is from 1 day to 3 days.

10. The transdermal patch of claim 8, wherein the period is from 12 hours to 24 hours.

11. The transdermal patch of claim 1, wherein the transdermal patch is a reservoir patch.

12. The transdermal patch of claim 11, wherein the reservoir patch includes a rate limiting membrane.

13. The transdermal patch of claim 1, wherein the active agent-containing composition contains from about 0.01 wt % and about 40 wt %.

14. The transdermal patch of claim 1, wherein the active agent-containing composition contains from about 0.05 wt % and about 30 wt %.

15. The transdermal patch of claim 1, wherein the second active agent is menthol.

16. The transdermal patch of claim 15, wherein the active agent-containing composition contains from about 1 wt % to about 20 wt % of methanol.

17. The transdermal patch of claim 1, wherein the second active agent is the vanilloid receptor-1 activator.

18. The transdermal patch of claim 17, wherein the vanilloid receptor-1 activator is selected from the group consisting of capsaicin, dihydrocapsaicin, nordihydrocapsaicin, homodihydrocapsaicin, homocapsaicin, resiniferatoxin, civamide, and combinations thereof.

19. The transdermal patch of claim 17, wherein the active agent-containing composition contains from about 0.01 wt % to about 10 wt % of the vanilloid receptor-1 activator.

20. The transdermal patch of claim 1, wherein the second active agent is salicylic acid or a salicylic acid derivative.

21. The transdermal patch of claim 20, wherein the second active agent is the salicylic acid derivative and is selected from the group consisting of salicylamide, sodium salicylate, diflunisal, niclosamide, acetyl salicylic aci, choline magnesium trialicylate, hydroxyethylsalicylate, diethylamine salicylate, triethylamine salicylate methyl salicylate and combinations thereof.

22. The transdermal patch of claim 20, wherein the active agent-containing composition contains from about 2 wt % to about 30 wt % of salicylic acid or a salicylic acid derivative.

23. A transdermal patch for the delivery of a nitric oxide-donor and a second active agent, comprising:
a backing layer, and
an active agent-containing composition supported at least in part by the backing layer, said active agent-containing composition comprising a nitric oxide-donor and a second active agent selected from the group consisting of an opioid, a local anesthetic, an NSAID, and combinations thereof, said active agent-containing composition containing between 1 wt % and 20 wt % of nitric oxide donor.

24. The transdermal patch of claim 23, wherein the nitric oxide donor is selected from the group consisting of nitroglycerin, isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof

25. The transdermal patch of claim 23, wherein the nitric oxide-donor is nitroglycerin.

26. The transdermal patch of claim 23, said transdermal patch having a drug delivery zone defined by the area where the active agent-containing composition contacts an intact human skin site, wherein the drug delivery zone has an area from about 2.5 cm² to 100 cm².

27. The transdermal patch of claim 23, said transdermal patch having a drug delivery zone defined by the area where the active agent-containing composition contacts an intact human skin site, wherein the drug delivery zone has an area from about 3 cm to about 50 cm².

28. The transdermal patch of claim 23, wherein the transdermal patch is an adhesive matrix patch.

29. The transdermal patch of claim 28, wherein the adhesive matrix includes an acrylic polymer.

30. The transdermal patch of claim 23, wherein the transdermal patch is formulated to deliver the active agents from the active agent-containing composition for a period of from 4 hours to 7 days.

31. The transdermal patch of claim 23, wherein the period is from 1 day to 3 days.

32. The transdermal patch of claim 23, wherein the period is from 12 hours to 24 hours.

33. The transdermal patch of claim 23, wherein the transdermal patch is a reservoir patch.

34. The transdermal patch of claim 33, wherein the reservoir patch includes a rate limiting membrane.

35. The transdermal patch of claim 23, wherein the active agent-containing composition contains from about 0.01 wt % and about 40 wt %.

36. The transdermal patch of claim 23, wherein the active agent-containing composition contains from about 0.5 wt % and about 30 wt %.

37. The transdermal patch of claim 23, wherein the second active agent is an opioid.

38. The transdermal patch of claim 37, wherein the opioid is selected from the group consisting of morphine, oxycodone, hydrocodone, codeine, diamorphine, dihydrocodeine, oxymorphone, nicomorphine, methadone, levomethadyl acetate hydrochloride, pethidine, fentanyl, alfentanil, sufentanil, remifentanil, ketobemidone, carfentanyl, propoxyphene, dextropropoxyphene, dextromoramide, bexitramide, piritramide benzomorphan derivatives, pentazocine, phenazocine, burprenorphine, butorphanol, nalbudine, dezocine, etorphine, tilidine, tramadol, loperamide, diphenoxylate, naloxone, naltrexone, and combinations thereof.

39. The transdermal patch of claim 37, wherein the active agent-containing composition contains from about 0.5 wt % and about 10 wt % of an opioid.

40. The transdermal patch of claim 23, wherein the second active agent is the local anesthetic.

41. The transdermal patch of claim 40, wherein the local anesthetic is selected from the group consisting of benzocaine, mepivacaine, ropivacaine, bupivacaine, lidocaine, prilocaine, procaine, chloroprocaine, EMLA, lignicaine, tetracaine, levobupivacaine, and combinations thereof.

42. The transdermal patch of claim 40, wherein the active agent-containing composition contains from about 0.5 wt % and about 10 wt % of a local anesthetic.

43. The transdermal patch of claim 23, wherein the second active agent is the NSAID.

44. The transdermal patch of claim 43, wherein the NSAID is selected from the group consisting of celecoxib, diclofenac potassium, diclofeniac sodium, diclofenac sodium with misprostol, diflunisal, etodolac, fenoprofen calcium, flurbiprofen, ibuprofen, indomethacin, ketoprofen, mclofenamate sodium, mefenamic acid, meloxicam, nabumetone, naproxen, naproxen sodium, oxaprozin, piroxicam, rofecoxib, salsalate, sulindac, tolmetin sodium valdecoxib, and combinations thereof.

45. The transdermal patch of claim 43, wherein the active agent-containing composition contains from about 3 wt % and about 25 wt % of an NSAID.

46. A transdermal patch for the delivery of a nitric oxide-donor and a second therapeutic agent, comprising:
a backing layer, and
an active agent-containing composition supported at least in part by the backing layer, said active agent-containing composition comprising a nitric oxide donor and a corticosteroid, said active agent-containing composition containing between 1 wt % and 20 wt % of nitric oxide donor.

47. The transdermal patch of claim 46, wherein the nitric oxide donor is selected from the group consisting of nitroglycerin, isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof

48. The transdermal patch of claim 46, wherein the nitric oxide donor is nitroglycerin.

49. The transdermal patch of claim 46, said transdermal patch having a drug delivery zone defined by the area where the active agent-containing composition contacts an intact human skin site, wherein the drug delivery zone has an area from about 2.5 cm² to 100 cm².

50. The transdermal patch of claim 46, said transdermal patch having a drug delivery zone defined by the area where the active agent-containing composition contacts an intact human skin site, wherein the drug delivery zone has an area from about 3 cm to about 50 cm².

51. A transdermal patch of claim 46, wherein the corticosteroid is selected from the group consisting of prednisone, prednisolone, beclomethasone, cortisone, dexamethasone, hydrocortinsone, methylprednisolone, triamcinolone, fludrocortisones, deflazacort, beclomethasone, dexamethasone, cortisol, and combinations thereof.

52. The transdermal patch of claim 46, wherein the active agent-containing composition contains from about 0.5 wt % and about 10 wt % of a corticosteroid.

53. The transdermal patch of claim 46, wherein the transdermal patch is an adhesive matrix patch.

54. The transdermal patch of claim 46, wherein the adhesive matrix includes an acrylic polymer.

55. The transdermal patch of claim 46, wherein the transdermal patch is formulated to deliver the active agents from the active agent-containing composition for a period of from 4 hours to 7 days.

56. The transdermal patch of claim 46, wherein the period is from 1 day to 3 days.

57. The transdermal patch of claim 46, wherein the period is from 12 hours to 24 hours.

58. The transdermal patch of claim 46, wherein the transdermal patch is a reservoir patch.

59. The transdermal patch of claim 46, wherein the transdermal patch includes a rate limiting membrane.

60. A transdermal patch for the delivery of a nitric oxide-donor and a second therapeutic agent, comprising:
a backing layer, and
an active agent-containing composition supported at least in part by the backing layer, said active agent-containing composition comprising a nitric oxide-donor and a vasoconstrictor, said active agent-containing composition containing between 1 wt % and 20 wt % of nitric oxide donor.

61. The transdermal patch of claim 60, wherein the nitric oxide donor is selected from the group consisting of nitroglycerin, isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof

62. The transdermal patch of claim 60, wherein the nitric oxide donor is nitroglycerin.

63. The transdermal patch of claim 60, said transdermal patch having a drug delivery zone defined by the area where the active agent-containing composition contacts an intact human skin site, wherein the drug delivery zone has an area from about 2.5 cm² to 100 cm².

64. The transdermal patch of claim 60, said transdermal patch having a drug delivery zone defined by the area where the active agent-containing composition contacts an intact human skin site, wherein the drug delivery zone has an area from about 3 cm to about 50 cm².

65. The transdermal patch of claim 60, wherein the vasoconstrictor is selected from the group consisting of epinephrine, norepinephrine, levonordefrin, felypressin, phenylephrine, metaraminol, flunarizine, hydroxynorephedrine, methoxamine HCl, pizotifen, propranolol, ergotamine, caffeine, sumatriptan succinate, and combinations thereof.

66. The transdermal patch of claim 60, wherein the active agent-containing composition contains from about 0.5 wt % and about 10 wt % of a vasoconstrictor.

67. The transdermal patch of claim 60, wherein the transdermal patch is an adhesive matrix patch.

68. The transdermal patch of claim 67, wherein the adhesive matrix includes an acrylic polymer.

69. The transdermal patch of claim 60, wherein the transdermal patch is formulated to deliver the active agents from the active agent-containing composition for a period of from 4 hours to 7 days.

70. The transdermal patch of claim 60, wherein the period is from 1 day to 3 days.

71. The transdermal patch of claim 60, wherein the period is from 12 hours to 24 hours.

72. The transdermal patch of claim 60, wherein the transdermal patch is a reservoir patch.

73. The transdermal patch of claim 72, wherein the transdermal patch includes a rate limiting membrane.

74. A method for delivering a nitric oxide-donor and a second active agent to a subject having a therapeutic need, comprising:
applying a transdermal patch to a skin surface, said transdermal patch including an active agent-containing composition comprising a nitric oxide-donor and a second active agent selected from the group consisting of menthol, a vanilloid receptor-1 activator, salicylic acid, an opioid, a local anesthetic, an NSAID, a corticosteroid, a vasoconstrictor, derivatives thereof, and mixtures thereof, said active agent-containing composition containing between 1 wt % and 20 wt % of nitric oxide donor.

75. The method of claim 74, wherein the nitric oxide donor is selected from the group consisting of nitroglycerin, isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof

76. The transdermal patch of claim 74, wherein the nitric oxide donor is nitroglycerin.

77. The method of claim 74, said transdermal patch having a drug delivery zone defined by the area where the active agent-containing composition is contacted by intact human skin site, wherein the drug delivery zone has an area from 2.5 cm² to 100 cm².

68. The method of claim 74, wherein the transdermal patch is a matrix patch.

78. The method of claim 74, wherein the transdermal patch is formulated to deliver the active agents from the active agent-containing composition for a period of from 4 hours to 7 days.

79. The method of claim 74, wherein the period is from 1 day to 24 days.

80. The method of claim 74, wherein the period is from 12 hours to 24 hours.

81. The method of claim 74, wherein the subject having the therapeutic need is experiencing pain and the transdermal patch is applied for reducing said pain.

82. The method of claim 74, wherein the subject having the therapeutic need has damaged tissue and the transdermal patch is applied for accelerating healing of the damaged tissue.

83. The method of claim 74, wherein the subject having the therapeutic need has tendinopathy and the transdermal patch is applied to improve function of an afflicted tendon.

84. The method of claim 74, wherein the subject having the therapeutic need has cancer and the transdermal patch is applied to inhibit metastasis.

85. The method of claim 74, wherein the subject having the therapeutic need is suffering from acute inflammation and the transdermal patch is applied to reduce said inflammation.

86. The method of claim 74, wherein the second active agent is menthol.

87. The method of claim 86, wherein the active agent-containing composition contains from about 1 wt % and about 20 wt % of menthol.

88. The method of claim 74, wherein the second active agent is the vanilloid receptor-1 activator.

89. The method of claim 88, wherein the vanilloid receptor-1 activator is selected from the group consisting of capsaicin, dihydrocapsaicin, nordihydrocapsaicin, homodihydrocapsaicin, homocapsaicin, resiniferatoxin, civamide, and combinations thereof.

90. The method of claim 88, wherein the active agent-containing composition contains from about 0.01 wt % and about 10 wt % of a vanilloid receptor-1 activator.

91. The method of claim 74, wherein the second active agent is salicylic acid or a salicylic acid derivative.

92. The method of claim 91, wherein the second active agent is the salicylic acid derivative and is selected from the group consisting of salicylamide, sodium salicylate, diflunisal, niclosamide, acetyl salicylic aci, choline magnesium trialicylate, hydroxyethylsalicylate, diethylamine salicylate, triethylamine salicylate methyl salicylate and combinations thereof.

93. The method of claim 91, wherein the active agent-containing composition contains from about 2 wt % and about 30 wt % of salicylic acid or salicylic acid derivative.

94. The method of claim 74, wherein the second active agent is the opioid.

95. The method of claim 94, wherein the opioid is selected from the group consisting of morphine, oxycodone, hydrocodone, codeine, diamorphine, dihydrocodeine, oxymorphone, nicomorphine, methadone, levomethadyl acetate hydrochloride, pethidine, fentanyl, alfentanil, sufentanil, remifentanil, ketobemidone, carfentanyl, propoxyphene, dextropropoxyphene, dextromoramide, bexitramide, piritramide benzomorphan derivatives, pentazocine, phenazocine, burprenorphine, butorphanol, nalbudine, dezocine, etorphine, tilidine, tramadol, loperamide, diphenoxylate, naloxone, naltrexone, and combinations thereof.

96. The method of claim 94, wherein the active agent-containing composition contains from about 0.5 wt % and about 10 wt % of an opioid.

97. The method of claim 74, wherein the second active agent is the local anesthetic.

98. The method of claim 97, wherein the local anesthetic is selected from the group consisting of, benzocaine, mepivacaine, ropivacaine, bupivacaine, lidocaine, prilocaine, procaine, chloroprocaine, EMLA, lignicaine, tetracaine, levobupivacaine, and combinations thereof.

99. The method of claim 97, wherein the active agent-containing composition contains from about 0.5 wt % and about 10 wt % of a local anesthetic.

100. The method of claim 74, wherein the second active agent is the NSAID.

101. The method of claim 100, wherein the NSAID is selected from the group consisting of celecoxib, diclofenac potassium, diclofeniac sodium, diclofenac sodium with misprostol, diflunisal, etodolac, fenoprofen calcium, flurbiprofen, ibuprofen, indomethacin, ketoprofen, mclofenamate sodium, mefenamic acid, meloxicam, nabumetone, naproxen, naproxen sodium, oxaprozin, piroxicam, rofecoxib, salsalate, sulindac, tolmetin sodium valdecoxib.

102. The method of claim 100, wherein the active agent-containing composition contains from about 3 wt % and about 25 wt % of an NSAID.

103. The method of claim 74, wherein the second active agent is the corticosteroid.

104. A method of claim 103, wherein the corticosteroid is selected from the group consisting of prednisone, prednisolone, beclomethasone, cortisone, dexamethasone, hydrocortinsone, methylprednisolone, triamcinolone, fludrocortisones, deflazacort, beclomethasone, dexamethasone, cortisol, and combinations thereof.

105. The method of claim 103, wherein the active agent-containing composition contains from about 0.5 wt % and about 10 wt % of a corticosteroid.

106. The method of claim 74, wherein the second active agent is a vasoconstrictor.

107. The method of claim 106, wherein the vasoconstrictor is selected from the group consisting of epinephrine, norepinephrine, levonordefrin, felypressin, phenylephrine, metaraminol, flunarizine, hydroxynorephedrine, methoxamine HCl, pizotifen, propranolol, ergotamine, caffeine, sumatriptan succinate, and combinations thereof.

108. The method of claim 106, wherein the active agent-containing composition contains from about 0.5 wt % and about 20 wt % of a vasoconstrictor.
